(19) United States
(12) Patent Application Publication
(10) Pub. No.: US 2017/0340272 A1
(43) Pub. Date: Nov. 30, 2017

(54) DECISION SUPPORT SYSTEM FOR CNS DRUG DEVELOPMENT

(71) Applicant: PAREXEL International Corporation, Waltham, MA (US)

(72) Inventors: Larry Ereshefsky, Marina Del Rey, CA (US); Brett English, Los Angeles, CA (US)

(73) Assignee: PAREXEL International Corporation, Waltham, MA (US)

(21) Appl. No.: 15/606,676

(22) Filed: May 26, 2017

Related U.S. Application Data
(60) Provisional application No. 62/342,818, filed on May 27, 2016.

(51) Int. Cl. A61B 5/00; G06F 19/00; G06N 3/06

(52) U.S. Cl. CPC A61B 5/4848; G06N 3/061; G06F 19/3437; G06F 19/3443; G06F 19/345

(57) ABSTRACT

A decision support tool for development of drugs targeting central nervous system conditions. The tool receives measurements made on subjects, which are converted to model outputs using neurocircuitry models. The models are used by a computing device to generate neuro-circuitry based signatures. Neuro-circuitry based signatures associated with an investigational compound may be compared to reference neuro-circuitry based signatures to identify parameters of a clinical trial protocol. The neuro-circuitry based signature comparisons, when generated based on measurement data collected in early phases of a clinical trial process, may increase the likelihood that the investigational compound will quickly and cost-effectively emerge from clinical trials with proof that the investigational compound is effective for treating one or more CNS conditions. The decision support tool may also indicate early phase measurements to make based on a condition against which an investigational compound is theorized to be effective against.

### RELATED APPLICATIONS

The present application claims the benefit under 35 U.S.C. §119(e) of U.S. Provisional Patent Application Ser. No. 62/342,818 titled “DECISION SUPPORT SYSTEM FOR CNS DRUG DEVELOPMENT,” filed May 27, 2016, which is incorporated herein by reference in its entirety.

### BACKGROUND

Central Nervous System (CNS) drugs have been developed to treat many human psychiatric and neurologic diseases, such as depression or dementia. These drugs have been developed according to a conventional drug development model, where novel compounds exhibiting favorable pre-clinical profiles are advanced to early clinical testing, largely based upon data from animal models.

Drugs that show promise in pre-clinical testing may then be advanced to small scale human trials, often in healthy volunteers who do not have the disease for which these novel compounds are therapeutically targeted. If the compound appears safe, it may be tested in humans with the targeted disease to determine a range of treatment conditions (e.g., doses of a compound) that appear clinically effective, at least for CNS indications, based on subjective endpoints. The treatment conditions established in these trials are then tested in much wider scale, controlled clinical trials that assess efficacy and safety of the drug relative to placebo or known alternative treatments.

The desired result of initiating a clinical trial is to achieve regulatory approval for marketing the drug for an intended indication. However, within CNS drug development, a number of novel compounds fail to successfully make it to market despite having well-developed pre-clinical data suggesting efficacy. These failures may arise from the complexity of the human brain and CNS as a whole and the heterogeneity of the various disorders. Some seemingly promising drug candidates reach wide scale clinical trials and fail to differentiate relative to placebo. About 90% of CNS drugs that enter the clinical trial process fail to achieve FDA approval. Further, it has been reported at the American Society for Experimental Neuro Therapeutics, 16th Annual Meeting, Feb. 20, 2014 Jill Heemskerk, PhD, Deputy Director, Division of Adult Translational Research, National Institute of Mental Health, NIH that “On average, a marketed psychiatric drug is efficacious in approximately half of the patients who take it. One reason for this low response rate is the artificial grouping of heterogeneous syndromes with different pathophysiological mechanisms into one disorder.”

### SUMMARY

Some aspects are embodied as a system for predicting the effectiveness of a study compound for treating a CNS condition if the study compound is administered to a patient with the CNS condition. The system comprises at least one processor configured to: access at least one reference neuro-circuitry based signature computed from measurements made on a first plurality of subjects having the CNS condition and treated with reference compounds for which the effectiveness on the CNS condition has been previously determined; receive measurement data collected during a plurality of measurements of a second plurality of subjects treated with the study compound. The system may further comprise at least one storage medium storing processor-executable instructions that, when executed by the at least one processor, perform a method comprising: generating from the measurement data a neuro-circuitry based signature associated with the study compound, the generated neuro-circuitry based signature comprising a plurality of model outputs computed using a plurality of neurocircuit models; and generating a result indicating effectiveness of the study compound for the condition based at least in part on comparing the generated neuro-circuitry based signature to the at least one reference neuro-circuitry based signature.

In some embodiments, the method further comprises generating, if the result indicates that the study compound is ineffective for treating the CNS condition, an indication of a second CNS condition to evaluate as part of a clinical trial of the study compound, and selecting a set of measurements that provide measurement data for applying a plurality of neurocircuit models associated with the second CNS condition. In some embodiments, selecting the set of measurements further comprises identifying a plurality of measurements that map to a reference neuro-circuitry based signature associated with the second CNS condition via the plurality of neurocircuit models. In some embodiments, generating the result includes generating a result indicating whether the study compound alleviates at least one symptom associated with the CNS condition. In some embodiments, the at least one reference neuro-circuitry based signature is representative of a response to the reference compound of at least one second subject that has the CNS condition. In some embodiments, the at least one reference neuro-circuitry based signature is indicative of alleviation of at least one symptom of the CNS condition in the at least one second subject in response to administering the reference compound to the second subject. In some embodiments, generating a result indicating effectiveness comprises comparing the generated neuro-circuitry based signature to the at least one reference neuro-circuitry based signature to compute a level of similarity between the neuro-circuitry based signature and the at least one reference neuro-circuitry based signature. In some embodiments, generating a result indicating effectiveness further comprises selecting a portion of the neurocircuits of the plurality of neurocircuits that correspond to brain functions indicative of the CNS condition and comparing model outputs associated with the portion of the neurocircuits between the neuro-circuitry based signature and the at least one reference neuro-circuitry based signature.

Another aspect may be embodied as a system for predicting at least one CNS condition treatable with a study compound if the study compound is administered to a patient with the at least one CNS condition. The system comprises at least one processor configured to: access at least one reference neuro-circuitry based signature computed from measurements made on a first plurality of subjects having a plurality of CNS conditions and treated with at least one reference compound that has been previously determined to be effective in treating a CNS condition; receive measurement data collected during a plurality of measurements of at least one second subject to whom the study compound has been administered. The system may also comprise at least one storage medium storing processor-executable instructions that, when executed by the at least one processor, perform a method comprising: generating a neuro-circuitry based signature associated with the study compound, the generated neuro-circuitry based signature comprising a plurality of model outputs generated by computing, using a plurality of neurocircuit models, the plurality of model outputs from the measurement data; and indicating at least one CNS condition by: comparing the generated neuro-circuitry based signature to the at least one reference neuro-circuitry based signature and, based on a degree of similarity between the generated neuro-circuitry based signature and a reference neuro-circuitry based signature of the at least one reference neuro-circuitry based signature associated with effective treatment of a known CNS condition, indicating that the study compound is predicted to be effective in treating the known CNS condition.

In some embodiments, the method further comprises identifying at least one domain of brain function affected by the compound by determining a portion of model outputs of the plurality of model outputs that describe the response of the subject to the compound based on the degree of similarity between the generated neuro-circuitry based signature and the reference neuro-circuitry based signature. In some embodiments, the method further comprises identifying at least one neurocircuit among the plurality of neurocircuits associated with the portion of model outputs. In some embodiments, the method further comprises identifying the at least one CNS condition based on the at least one domain of brain function. In some embodiments, comparing the generated neuro-circuitry based signature to the at least one reference neuro-circuitry based signature comprises computing a correlation value between the generated neuro-circuitry based signature and the at least one reference neuro-circuitry based signature and comparing the correlation value to a threshold.

Yet another aspect may be embodied as a system for designing a clinical trial for a study compound. The system may comprise at least one processor configured to: access at least one reference neuro-circuitry based signature computed from a plurality of measurements made on a first plurality of subjects having a CNS condition and treated with reference compounds that have previously been shown to be effective at treating a CNS condition, wherein each of the at least one reference neuro-circuitry based signature has associated therewith a plurality of neurocircuit models from which the reference neuro-circuitry based signature is generated based on the plurality of measurements; receive a study neuro-circuitry based signature representative of a second plurality of subjects' response to the study compound, wherein the study neuro-circuitry based signature includes a plurality of model outputs computed, using neurocircuitry models, from measurements made on the second plurality of subjects before and after treatment with the study compound. The system may also comprise at least one non-transitory storage medium storing processor-executable instructions that, when executed by the at least one processor, perform a method comprising: comparing the study neuro-circuitry based signature to the at least one reference neuro-circuitry based signature; based on a degree of similarity between the study neuro-circuitry based signature and a selected reference neuro-circuitry based signature of the at least one reference neuro-circuitry based signature, selecting a set of measurements that provide measurement data for applying the plurality of neurocircuit models associated with the selected reference neuro-circuitry based signature.

In some embodiments, the method further comprises generating a result indicating at least one CNS condition to evaluate as part of the clinical trial. The at least one CNS condition is associated with at least one domain of brain function. In some embodiments, the method further comprises generating a result indicating at least one symptom to identify in subjects as part of enrollment in the clinical trial. The at least one symptom is associated with the at least one CNS condition. In some embodiments, the method further comprises identifying at least one type of equipment for obtaining the measurement data during the clinical trial.

A further aspect may be embodied as a system for designing a clinical trial. The system may comprise at least one processor configured to: receive user input indicating a CNS condition to evaluate as part of the clinical trial; and access a plurality of reference neuro-circuitry based signatures. The system may further comprise at least one storage medium storing processor-executable instructions that, when executed by the at least one processor, perform a method comprising: selecting at least one reference neuro-circuitry based signature of the plurality of reference neuro-circuitry based signatures, the at least one selected reference neuro-circuitry based signature being computed from a plurality of measurements made on a first plurality of subjects having the CNS condition and treated with reference compounds for which the effectiveness on the condition has been previously determined, wherein each of the at least one reference neuro-circuitry based signature has associated therewith a plurality of neurocircuit models that map the plurality of measurements to the reference neuro-circuitry based signature; and generating, based on the plurality of measurements mapped to the at least one reference neuro-circuitry based signature via the neurocircuit models of the at least one selected reference neuro-circuitry based signature, a result indicating a plurality of measurements to include in a protocol for the clinical trial.

In some embodiments, the method further comprises identifying a portion of the neurocircuit models of the plurality of neurocircuit models that correspond to brain functions indicative of the CNS condition. In some embodiments, the plurality of measurements to include in the protocol of the clinical trial provide data indicative of the brain functions.

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic of an exemplary system for analyzing data from clinical trials and/or designing clinical trials.

FIG. 2 illustrates an exemplary schematic of a data flow process used to determine a neuro-circuitry based signature performed by decision support tool.

FIG. 3A is a schematic representation of a database that relates different conditions to model outputs from neurocircuit models.

FIG. 3B is a schematic representation of a database that relates different compounds to model outputs from neurocircuit models.

FIG. 4 is a schematic illustration of phases of evaluating an investigational drug.

FIG. 5 is a flowchart of an exemplary method for designing a clinical trial to evaluate a compound.

FIG. 6 is a flowchart of an exemplary method for determining the effectiveness of a compound in treating a CNS condition.

FIG. 7 is a block diagram of an exemplary computer system on which some embodiments may be implemented.

### DETAILED DESCRIPTION

The inventors have recognized and appreciated that, by exploiting neurocircuitry-based signatures, the time and cost of CNS drug development may be decreased, while increasing the likelihood that in clinical trials an investigational drug will be proven effective for its intended indication. Neurocircuitry-based signatures may be exploited in a decision support tool that may be applied at one or more stages of the drug development process. The neurocircuitry-based signatures may allow for incorporation of the complexity of human cognitive/behavioral neuroscience in investigating a human disease state and/or the effects of a drug on the human disease state. Further, a neurocircuitry-based approach may provide greater sensitivity and precision in detecting an effect of a drug than a clinical assessment. These techniques may improve interpretation of data from clinical trials at different phases of drug development.

The decision support tool, for example, may:
predict whether a compound (e.g., investigational drug) is likely to be effective for specific indications based on the neuro-circuitry based signature of the compound.
predict indications that a compound is likely to be effective against based on the compound's influence on neurocircuitry related to a neurological disorder or disease.
guide a researcher in structuring measurements to make at one stage of drug development to collect data that will enable reliable decisions about other stages.
identify additional pre-clinical measurements that will reveal a finding or measurements that, if made during a clinical trial, will provide a high degree of discrimination between an effective and ineffective drug.
identify individuals that, if enrolled as subjects in a clinical trial, will provide data that provides a high degree of discrimination between an effective and ineffective drug or otherwise suggesting enriched patient populations.
guide selection of appropriate doses for treating individuals or populations.

These and other decisions may be made with one or more neuro-circuitry based signatures, each of which may be derived from a combination of model outputs computed, using a neurocircuit model, from one or more measurements made on one or more subjects.

Neurocircuitry constructs have been developed by linking brain regions and information processing circuits to observable behavior or cognitive function (e.g., attention, perception, working memory). For example, a neurocircuit may relate firing of neurons in a specific region of a subject's brain to attention or focus on a task. Each neurocircuit is a construct that classifies brain functioning with respect to behavior or cognition, and can be rated along a spectrum (e.g., above or below a statistical norm for an individual or population by a certain degree). As used herein, “neurocircuits” are based on a model of brain function developed by the National Institute of Mental Health (NIMH).

A neurocircuit may relate measurable characteristics of a subject to an aspect of brain function. That relationship may be expressed in computer-executable form as a “neurocircuit model.” A model output from a “neurocircuit model” may provide a result indicating the extent to which an aspect of a subject's brain functioning is active. The model output may be a specific value representative of activity or may represent a probability of a specific activity level, or may quantify brain functioning in any other suitable way. The relation between measurable characteristics and the model output may, but need not, be tied to function or structure of a specific region of the brain. A “neurocircuit model” may define how to compute a model output representing the degree to which a subject exhibits a particular aspect of brain function. In the example of assessing focus, the model may define how to compute a model output, indicative of a degree of attention being paid by a subject, based on one or more measurements of neural activity in a region of the brain.

As used herein, a “neurocircuit model” defines a computation relating data collected as part of one or more measurements to a model output reflecting position along a spectrum of brain activity that a neurocircuit represents. In some embodiments, these model outputs may be expressed in a format that allows automated comparisons between model outputs or collections of model outputs representing neuro-circuitry based signatures. Each model output, for example, may be expressed as a number, including a fraction or a percentage, representing positioning along that spectrum. As another example, each model output may be expressed as one of an enumerated set of ordered values. In such an embodiment, a comparison between two neuro-circuitry based signatures may be expressed as the difference in positions in the order of the values assigned to those neuro-circuitry based signatures. The neurocircuit model may be expressed as a collection of computer-executable instructions written in any suitable language or captured in any suitable form. Such models may be developed based on scientific literature and/or prepared experimentally, or derived in any other suitable way. Any suitable neurocircuit models and measurements, whether now known or hereafter developed, may be used to form a neuro-circuitry based signature related to a predicted efficacy.

Specific technologies to perform measurements can be used to assess constructs within a specific individual exist, and, depending on the neurocircuit model, may involve diagnostic tests, including electroencephalography (EEG), functional imaging, behavioral measurements, and/or clinical observation, as well as other measurements. In the example above in which an output of a model representing a degree of attention is computed based on a measurement of neural activity, the measurements input to the neurocircuit model may be acquired with an fMRI or EEG machine. Many measurements are known in the art for generating data that might be an input to a neurocircuit. However, it should be appreciated that “measurement” includes, not just data collected with a sensing device, but also data collected in other ways, including by accessing public domain information, such as publications or on the World Wide Web.

A CNS condition of a subject may be represented by model outputs from multiple neurocircuit models. In this context, a CNS condition includes conditions that are sometimes referred to in the art as neurological conditions or are sometimes referred to as psychiatric conditions. Moreover, a condition may represent a diagnosis, phenotype, syndrome, symptom or any other hierarchical categorization of function or lack of function of a subject.

The model outputs of the neurocircuit models may be combined into a “neuro-circuitry based signature.” As used herein, a “neuro-circuitry based signature” is a representation of a combination of model outputs that were, in turn, computed, using neurocircuit models, from multiple measurements made on one or more subjects. The neuro-circuitry based signature may be associated with a patient, population, compound, condition or other entity. In some scenarios, the measurements used to compute the signature may be collected as part of a trial or under other circumstances in which the signature may be analyzed to reveal a state or response of an entity. In other scenarios, a neuro-circuitry based signature may be computed from measurements in which the state or response of the entity is known, and the signature may serve as a “reference neuro-circuitry based signature.” By comparing a neuro-circuitry based signature formed from measurements under an experimental condition to one or more reference neuro-circuitry based signatures, information about the entity for which the neuro-circuitry based signature was formed might be inferred. When there is a sufficiently high correlation between the neuro-circuitry based signature and the reference neuro-circuitry based signature, the entity represented by the neuro-circuitry based signature may be inferred to have the state or exhibit the response associated with the reference neuro-circuitry based signature.

Any suitable number of the model outputs yielded by such neurocircuit models may be combined to create a neurocircuitry-based signature to characterize some entity, such as a subject, a population, or a drug. The model outputs may be combined with each other, and in some embodiments, other information, to create the neuro-circuitry based signature. The specific method by which neurocircuit model outputs are combined to create a neuro-circuitry based signature may depend on the neurocircuit models and the nature of the neuro-circuitry based signature. However, in some embodiments, the number of neurocircuit models, and therefore the number and type of measurements performed on a subject to create a neuro-circuitry based signature, may be selected so as to increase the discriminatory capability of that neuro-circuitry based signature. As a specific example, when testing a drug designed for improved mental focus, a signature may be created from model outputs of neurocircuit models for neurocircuits associated with focus and cognition.

When the neuro-circuitry based signature represents an individual at a single instance in time, the neuro-circuitry based signature may be a multi-dimensional value. In some embodiments, the neuro-circuitry based signature may be the concatenation or other combination of neurocircuitry-based model outputs obtained contemporaneously, while the subject is in the same state. It is not necessary that these model outputs be weighted the same when combined into a signature. In the example of a signature for testing a drug intended to improve focus, the model output associated with a neurocircuit representing focus may be more heavily weighted than other model outputs combined in the neuro-circuitry based signature.

In other embodiments, the neuro-circuitry based signature may represent a population. For example, the neuro-circuitry based signature may include model outputs computed for multiple subjects, who may be selected based on a common characteristic or set of characteristics, such as being assigned to the same treatment group in a clinical trial or having been diagnosed with the same CNS condition. Neurocircuit model outputs computed from measurements made on multiple subjects may be combined arithmetically, such as by computing an average of the model outputs. These model outputs alternatively may be combined statistically, with the combination representing a range of values associated with a norm. Moreover, other types of analysis may be made to derive a model output, particularly for a population, including probabilistic, population Bayesian estimates, and a variety of multi-factorial and cluster analyses.

A neuro-circuitry based signature alternatively or additionally may represent a compound, such as a study drug. The compound may influence particular neurocircuitry such that a combination of model outputs from subjects having been administered the compound may act as a neuro-circuitry based signature for the compound. Accordingly, a neuro-circuitry based signature of a subject or population that has been exposed to a drug may act as a neuro-circuitry based signature for the drug. To represent a drug, the neuro-circuitry based signature may be computed from measurements made on multiple subjects after administration of the drug, while the drug is affecting the subjects' CNS. Likewise, a neuro-circuitry based signature of a subject or population of subjects with a specific condition may be a neuro-circuitry based signature of the condition.

In other embodiments, a neuro-circuitry based signature may represent a comparison between two or more subject states. The neuro-circuitry based signature may indicate a change in one or more neurocircuitry-based model outputs computed based on measurements made on the subject in different states. The “state” may represent any suitable state that is being studied or relevant to a study. For example, the “state” may be that the patient has been diagnosed with a particular condition, has been given a particular drug, has a particular concentration of drug in their bloodstream, or any other suitable state. As a specific example, a neuro-circuitry based signature may reflect a change in model outputs based on measurements performed on a subject before and after administration of an investigational drug. Such a neuro-circuitry based signature may provide information indicative of the subject's response to the drug. Such a neuro-circuitry based signature may act as a neuro-circuitry based signature of the subject, indicating the subject's response to the drug, but may also act as a neuro-circuitry based signature of the drug.

A neuro-circuitry based signature representing a comparison of states may be formatted in any suitable way. In some embodiments, the neuro-circuitry based signature may include model outputs in multiple states. In other embodiments, the neuro-circuitry based signature may reflect measurements made with the subject in different states by capturing values representing change from one state to another or a trend in model outputs across multiple states.

Regardless of the form of the neuro-circuitry based signature, a neuro-circuitry based signature may be compared to other neuro-circuitry based signatures that serve as reference neuro-circuitry based signatures. Such a comparison may generate information to guide decision-making in a clinical trial. The reference neuro-circuitry based signatures, like neuro-circuitry based signatures described above, may be computed from measurements on an individual or a population. In some embodiments, the reference neuro-circuitry based signatures may be associated with state information indicating a state or states under which the measurements used to make the reference neuro-circuitry based signature were obtained. For example, the reference neuro-circuitry based signature may be associated with state information indicating that the subjects from whom the reference neuro-circuitry based signature was generated had a particular CNS condition or were treated with a particular drug. As other examples of state information, the reference neuro-circuitry based signatures may be associated with information indicating that the subjects did or did not respond to a particular drug. As yet a further example, state information may relate to the impact of a compound on a subject or population of subjects, including information such as pharmacokinetic, genomics, PK/PD modeling such as population pharmacokinetics and CNS-peripheral distribution estimates. In some embodiments, a system as described herein may assist in dose selection. In order to accomplish this, robust modeling linking measurable data with exposure may be employed. Those modeled results may be associated with either a neuro-circuitry based signature or a reference neuro-circuitry based signature, indicating a condition under which the measurements used to generate the signature were collected. In such an embodiment, a neuro-circuitry based signature selected because it indicates a highly effective treatment may be associated with information indicating an appropriate dose.

The nature of the decisions to be made may indicate the nature of the reference neuro-circuitry based signature, including the state information associated with the reference neuro-circuitry based signature. For example, when the reference neuro-circuitry based signatures are used to assess whether a study compound is likely to be effective in treating a CNS condition, the reference neuro-circuitry based signatures may include neuro-circuitry based signatures of other drugs determined to be effective and/or not effective in treating that CNS condition.

In some embodiments, the reference neuro-circuitry based signatures may be based on model outputs for the same neurocircuits that are used to generate a neuro-circuitry based signature being analyzed by comparison to the reference neuro-circuitry based signature. However, it should be appreciated that comparisons need not be limited to neuro-circuitry based signatures of identical format. In some embodiments, neuro-circuitry based signatures may be compared by considering only a common subset of neurocircuits reflected in the neuro-circuitry based signatures to be compared. Moreover, it is not required that comparisons identify exact matches between neuro-circuitry based signatures. For example, statistical measures of similarity may be used to identify matching neuro-circuitry based signatures or the likelihood of a match, which may then be compared to a threshold indicating a sufficiently high likelihood to deem the neuro-circuitry based signature a match to the reference neuro-circuitry based signature.

Moreover, it is not a requirement that the “reference neuro-circuitry based signature” be formatted like a neuro-circuitry based signature, with specific values or probabilities. In some embodiments, the reference neuro-circuitry based signature may be expressed as criteria defining a neuro-circuitry based signature matching the reference neuro-circuitry based signature. In some instances, the absolute value of model outputs for specific neurocircuit models may be unimportant, but the relative values of two or more of the model outputs may define a neuro-circuitry based signature of interest. Accordingly, the reference neuro-circuitry based signature to which neuro-circuitry based signatures are to be compared may define relative levels of two or more model outputs present in the neuro-circuitry based signature, and a match may be detected based on those model outputs having the relative levels. Likewise, when the neuro-circuitry based signature is based on measurements made over time, the change in values of certain model outputs may define neuro-circuitry based signatures of interest, and the reference neuro-circuitry based signature may be formatted as a specification of change over time that matches the reference. Accordingly, though examples are shown in which reference neuro-circuitry based signatures include a plurality of fields comprising values derived from neurocircuit model outputs, it should be appreciated that the reference neuro-circuitry based signature may be defined in other ways, such as a collection of conditions or a set of rules.

The neuro-circuitry based signatures, including reference neuro-circuitry based signatures, may reflect the state of the subject or subjects at a single time or may represent the subject or subjects while in a specific state. Alternatively, the reference neuro-circuitry based signature may represent a change in the subject or subjects over time or as the subject or subjects transition between states. For example, a reference neuro-circuitry based signature may represent a response of a subject that has a CNS condition to a reference compound by incorporating model outputs based on measurements made before and after the subject is treated with the reference compound. In some embodiments, a reference neuro-circuitry based signature may include model outputs showing differences relative to a healthy individual or average of individuals. For example, values constituting the reference neuro-circuitry based signature may represent a difference between a neuro-circuitry based signature representative of a subject with the condition and a neuro-circuitry based signature representative of a healthy individual. The reference neuro-circuitry based signature may be stored as values characterizing these responses or differences.

When the reference neuro-circuitry based signature is based on multiple instances of measurement data, collected for different subjects and/or at different times or in different states, the reference neuro-circuitry based signature may be formatted as an aggregation of this data or may be formatted as disaggregated data, which may be aggregated when the reference neuro-circuitry based signature is used. For example, when the neuro-circuitry based signature is based on measurements made over time, the change in values of certain model outputs may be stored as the reference neuro-circuitry based signature or the values for different times may be stored.

Alternatively or additionally, the reference neuro-circuitry based signature may be stored as part of a database that has neuro-circuitry based signatures based on model outputs in different states, such as of healthy subjects and subjects with the CNS condition or model outputs computed from measurements made before and after treatment with a drug. In such embodiments, comparison of a neuro-circuitry based signature to a reference neuro-circuitry based signature may entail analyzing the neuro-circuitry based signature and the information in the reference neuro-circuitry based signature database to determine whether the neuro-circuitry based signature exhibits the same change or pattern of change between states as the reference neuro-circuitry based signature.

Reference neuro-circuitry based signatures may include neuro-circuitry based signatures corresponding to any state or combination of states, such as neuro-circuitry based signatures corresponding to different types of compounds. The reference neuro-circuitry based signatures may be representative of the biological effects different compounds may have on an individual. Such reference neuro-circuitry based signatures may be derived from drugs that have known effects. These reference neuro-circuitry based signatures may be obtained by administering a drug to a group of people, obtaining measurement data indicative of the effect of the drug, and generating a neuro-circuitry based signature based on the measurement data applied to one or more neurocircuit models. The one or more neurocircuit models may represent brain functions demonstrated to be affected by the drug.

The ability to compare neuro-circuitry based signatures generated from measurements made on subjects to reference neuro-circuitry based signatures enables a computerized decision support tool that may be used in designing or managing a clinical trial. The tool may process data collected at any stage of the clinical trial process and guide decisions at either earlier or later stages in the clinical trial process. Decisions relating to later stages may guide the design or conduct of the trial, including futility measures whereby a study might be prematurely discontinued. Decisions at an earlier stage may reveal data that can be collected to validate findings already made, for example.

A use of such a tool may be to analyze the results of measurements performed for an investigational drug. In some embodiments, the tool may collect data based on measurements of one or more subjects given the investigational drug. The data may be used to generate a neuro-circuitry based signature for the drug by aggregating the model outputs for multiple test subjects computed with specific neurocircuit models. The neuro-circuitry based signature may then be compared to one or more reference neuro-circuitry based signatures. The reference neuro-circuitry based signatures may be derived from measurements of subjects given existing drugs with known pharmacology. However, it should be appreciated that the reference neuro-circuitry based signatures may be developed in other ways, such as by analyzing measurements on classes of drugs that have a desired effect to develop a hypothetical neuro-circuitry based signature that has a high degree of correlation with actual neuro-circuitry based signatures for drugs in that class, which may act as a reference neuro-circuitry based signature. Alternatively or additionally, a reference neuro-circuitry based signature may be developed based on theory or other analysis.

Regardless of how the reference neuro-circuitry based signatures are generated, the neuro-circuitry based signature comparisons may be used in any of multiple ways. As another example of how the comparisons might be used, based on similarities between neuro-circuitry based signatures of subjects given an investigational drug and one or more reference neuro-circuitry based signatures corresponding to a drug of known efficacy for a specific indication, a predicted efficacy of the investigational drug for a specific indication may be assessed. Such a comparison and prediction of efficacy may be made at one stage in a drug development process, and may then be used to guide later stages in the clinical development.

In some embodiments, the neuro-circuitry based signature of an investigational drug in a preclinical phase of drug development may be compared to reference neuro-circuitry based signatures. At this phase, the prediction may guide selection of an indication for which to test an investigational drug in clinical trials. The prediction may alternatively guide selection of additional pre-clinical testing to evaluate comparisons with reference neuro-circuitry based signatures. For example, a neuro-circuitry based signature with a first format may be formed, using a first set of neurocircuit models, during pre-clinical testing of an investigational compound. Based on a comparison to reference neuro-circuitry based signatures, that investigational compound may be predicted to be useful in treating a condition. Other reference signatures may be analyzed to determine that model outputs of a neurocircuit model not in the first set of neurocircuit models changes significantly in subjects having that condition who are effectively treated with a known drug. Additional testing may be specified, as part of pre-clinical or clinical testing, to ensure that measurements are available to compute model outputs of those highly discriminatory neurocircuit models. In this way, the set of neurocircuit models used to compute a neuro-circuitry based signature may be expanded or altered to better assess an investigational drug that is expected to exhibit the same efficacy. In some embodiments, a prediction made at the pre-clinical stage of what conditions an investigational compound may be effective against may guide the selection of other parameters of a trial, such as which measurements are made during a Phase I trial. Data gathered in a Phase I clinical trial may then be used to assess effectiveness for that indication.

As a specific example of how selecting testing to perform may improve overall drug development, Phase I trials are conventionally used to validate the safety and tolerability and to determine the pharmacokinetic properties of a medication. However, by appropriately selecting measurements to be performed in Phase I, data may be available to predict whether the investigational drug will be effective against its intended indication or to predict an indication against which it is likely to be effective. If the developmental compound is not predicted to be useful against any desired indication, further testing may be avoided, saving the expense of conducting further clinical trials. Alternatively or additionally, pre-clinical information, incorporating drug PK exposure considerations with central nervous system effects may be used to streamline the selection of a dose titer or otherwise structure later stages of a trial.

More generally, if the investigational drug is predicted to be effective against a desired indication, that drug may be tested for that indication in a later stage of development. That indication may again be the basis for selecting measurements to be performed during later phase clinical trials. Other parameters of the trial, such as a patient population on which to test the investigational drug, may also be selected based on a predicted efficacy. As a result, the data collected may reliably predict whether an investigational drug should proceed to the next trial phase for its intended indication. Alternatively, the data collected may provide a reliable prediction that the investigational drug should proceed with clinical trials for a different indication.

This approach may be applied at any stage of the development process. For example, it may be used in structuring Phase II trials, and analyzing the data collected during Phase II to determine whether and how a Phase III trial might be conducted. As another example, this approach may lead to the optimization of clinical trials to incorporate adaptive design and seamless phase 2-3 trials.

To support planning and conducting drug development activities, in accordance with some embodiments, the decision support tool may comprise a computing platform with at least one computing device configured to generate and compare neuro-circuitry based signatures and to output predictions, measurement information or other information that may control aspects of, or be used in, CNS drug development. Such a decision support tool may include at least one database of reference neuro-circuitry based signatures, such as neuro-circuitry based signatures derived from drugs that have known effects. The computing platform may identify matches between the neuro-circuitry based signature of a test drug and the known drugs in the database. A further database may indicate specific neurocircuits to assess for experimental drugs, based on intended indication for the drug or other parameters.

Further, the decision support tool may have access to one or more databases holding results of measurements performed on subjects given investigational drugs. Such access may be provided in any suitable way. In some embodiments, for example, one or more processors implementing the decision support tool may interface over a computing network to an electronic data capture (EDC) system or other data collection system in which results of such measurements may be stored. However, in some embodiments, the data may be received from or accessed directly from laboratories or other locations where the measurements are made.

One or more processors implementing the decision support tool may be programmed to access that data and generate a neuro-circuitry based signature for the investigational drug. The generated neuro-circuitry based signature may then be compared to neuro-circuitry based signatures in the database of reference neuro-circuitry based signatures to identify an indication against which the investigational drug is likely to be effective. The predicted indication may be output to a user of the tool or may be used in other ways, such as to select measurements to be performed in a later stage of a clinical trial.

The inventors have recognized and appreciated that, for any given indication, measurements associated with different neurocircuits or different combinations of neurocircuits provide different degrees of discrimination. Accordingly, one use of a decision support tool may be to compute and output, based on an intended indication of a drug, a result indicating measurements to be performed to form a neuro-circuitry based signature that will provide a high likelihood of distinguishing an investigational drug useful for the intended indication from one that would not be effective.

It should be appreciated that some or all of the actions described herein may be performed in whole or in part based on user input. Accordingly, a decision support tool may have one or more user interfaces. These interfaces may be configured to receive input, such as a desired indication for an investigational drug. These interfaces may alternatively or additionally be configured to provide output, such as a predicted indication for which a study drug will be effective or a suggested trial protocol.

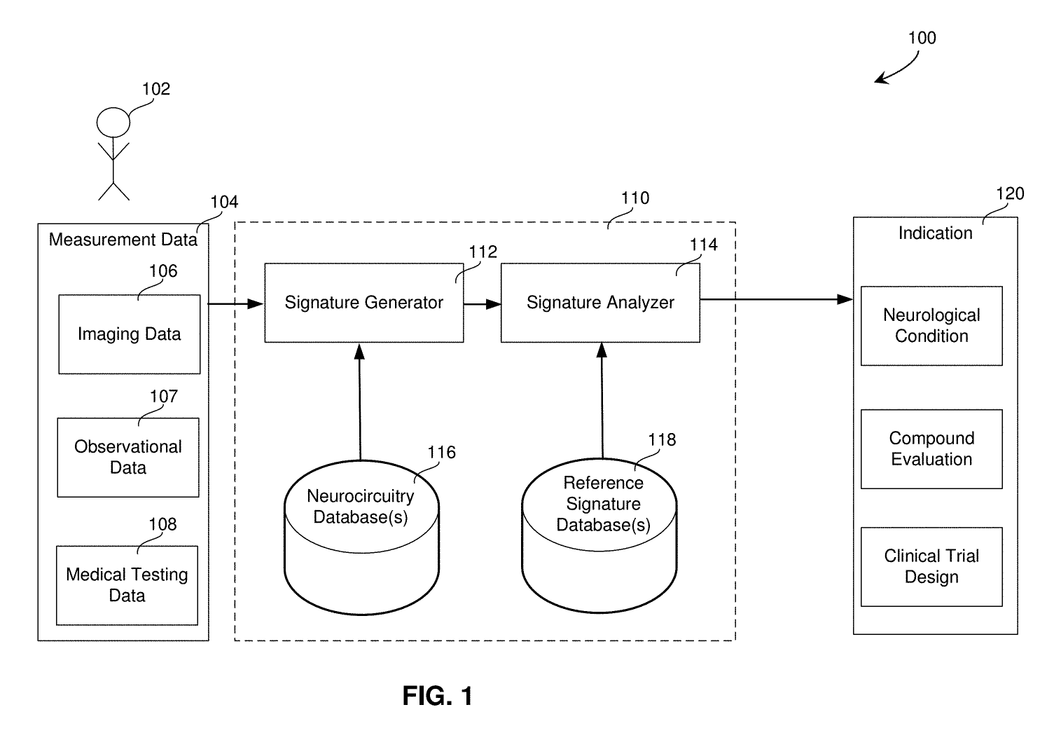

FIG. 1 illustrates an exemplary embodiment of system 100 for analyzing data from clinical trials and/or designing clinical trials. System 100 includes decision support tool 110 which is configured to receive measurement data 104 for one or more patients 102, which may include measurement data for a group of patients, generate and analyze a neuro-circuitry based signature based on the measurement data, and output indication result 120 of the analysis.

Measurement data 104 may include any suitable type of data acquired based on interactions with patient 102. Measurement data 104 may be obtained through any suitable techniques used to evaluate a person's behavioral and/or cognitive functions. FIG. 1 illustrates an embodiment in which measurement data 104 is obtained from three sources. However, measurement data 104 may be obtained from any number or types of sources. As depicted in FIG. 1, measurement data 104 may include imaging data 106, observational data 107, and/or medical testing data 108. Though not expressly illustrated, the observational data may be generated by a healthcare provider, but the invention is not so limited. In some embodiments, data may be entered by a subject, such as in response to a series of questions or to puzzles or challenges presented through an interface of a computer and designed to assess the subject's attention, reaction time or make other measurements that are useful in computing a model output. As further examples, measurements may include electrophysiological measurements, including event related potential measurements, biomarkers measured by a variety of analytic methods in a variety of biologic fluids, etc. Regardless of how collected, measurement data 104 may be captured and stored in one or more computing devices and/or storage devices.

Imaging data 106 may include data obtained from one or more types of imaging machines used to acquire images of a person's brain, which may reveal structural or functional characteristics of the brain. Examples of imaging techniques that may be used to acquire imaging data 106 may include magnetic resonance imaging (MRI), functional MRI (fMRI), arterial spin labeling (ASL) imaging, diffusion tensor imaging (DTI), resting state fMRI, positron emission tomography (PET), and fluorodeoxyglucose PET (FDG-PET). In preparing a neuro-circuitry based signature, imaging data 106 may be collected using one or multiple imaging techniques.

The image data may be processed in any suitable way to prepare it as an input to a neurocircuitry model. For example, acquired images may demonstrate activity levels for certain regions in a person's brain. In some embodiments, imaging data 106 may include image files acquired using one or more imaging techniques. Alternatively or additionally, imaging data 106 may include data derived by processing images, such as data identifying the size, shape or position of certain brain structures or regions and/or an activity level for an identified brain region. Imaging data 106 may include indications of anomalies (e.g., a tumor) identified by an imaging technique.

Imaging data may be supplied to decision support tool 110 in any suitable way. In some embodiments, an imaging machine that collects image information as the source of imaging data 106 may be located in one or more locations remote from computing equipment configured to implement decision support tool 110. In such an embodiment, the data may be communicated over one or more computer networks, including a wired and/or wireless network.

Moreover, it should be appreciated that, though not shown in FIG. 1 for simplicity, one or more manual or automated image “reading” processes may be performed on acquired images to generate imaging data 106. These processes may include comparing images to detect changes in one or more parameters, segmenting images to identify regions or structures and/or measuring characteristics of the identified regions, structures or other aspects of the image. Processing may include tractography, connectivity analyses, including default mode network, and task-based activations. This processing may be performed in or input through a separate computing device (not shown). However, in other embodiments, processing may alternatively or additionally be performed in the same computing device or devices that implement decision support tool 110.

Observational data 107 may include data obtained from one or more clinical techniques used to evaluate a patient's behavior and/or cognitive condition. Observational data 107 may include data obtained from patient interviews, patient's family history, a clinician's observations from interacting with a patient, computerized challenges or tests executed on a subject's computer or smartphone, cognitive assessments, and/or behavioral assessments.

In accordance with some embodiments, observational data 107 may be obtained, for example, by a clinician and/or devices that record parameters of human behavior, such as accelerometers that can be operated to detect gross bodily motion, an eye tracker that can be operated to detect focus or attention, a microphone that may be used to detect speech, and biomarkers in both biologic fluids and tissue samples derived from appropriate laboratory equipment, or any other suitable device. The recorded parameters of human behavior may be analyzed to detect stress or other psychological states. When observational data 107 is collected by a device, the device may be electronically coupled to decision support tool 110. Alternatively or additionally, observational data 107 may be entered by a human through a computer user interface or input into decision support tool 110 in any other suitable way. Medical testing data 108 may include data obtained from performing medical tests on a biological sample from a patient and/or on the patient. Medical testing data 108 may include data from electrophysiology measurements (e.g., QEEG, ERP, and PSG), blood tests, genetic tests, urinalysis, and any other suitable type of medical test to evaluate a property of a subject's condition. This data may be coupled from a device collecting the data to decision support tool 110. Alternatively or additionally, the medical testing data 108 may be input by a human into decision support tool 110 through a user interface or in any other suitable way.

Regardless of the manner in which measurement data 104 is input into decision support tool 110, decision support tool 110 may be configured to process that data and generate one or more types of outputs. That processing may entail generating one or more neuro-circuitry based signatures. As depicted, exemplary decision support tool 110 includes neuro-circuitry based signature generator 112 and neuro-circuitry based signature analyzer 114. Each of these processing components of decision support tool 110 may be implemented in software, hardware, or a combination of software and hardware. Components implemented in software may comprise sets of processor-executable instructions that may be executed by the one or more processors of decision support tool 110 to perform the functionality described herein. Any combination of neuro-circuitry based signature generator 112 and neuro-circuitry based signature analyzer 114 may be implemented on one or more separate machines, or parts of any or all of the components may be implemented across multiple machines in a distributed fashion and/or in various combinations.

Neuro-circuitry based signature generator 112 may receive one or more types of measurement data 104 and generate a neuro-circuitry based signature based on the measurement data and one or more neurocircuit models. The neuro-circuitry based signature may include information about the extent to which the measurement data indicates operation of one or more neurocircuits along a spectrum, such as the spectrum between normal and abnormal behavior. Decision support tool 100 may include neurocircuitry database(s) 116, which store neurocircuitry models associated with one or more neurocircuits. Each of the models may include information, which may be executable by a computing device, indicating how to compute a model output from the one or more pieces of the measurement data in accordance with one or more neurocircuit models. In the example of a study being used to assess effectiveness of an investigational compound in improving focus, the measurements may be applied as inputs to neurocircuit models selected to form a signature useful in studying focus. However, it should be appreciated that the specific neurocircuit models used in generating the signature may be selected based on the desired insights to be derived from a study being conducted or in any other suitable way.

In some embodiments, neurocircuitry database 116 may contain multiple models and neuro-circuitry based signature generator 112 may generate a neuro-circuitry based signature by combining the model outputs produced by all of the models in neurocircuitry database 116. In such an embodiment, for example, neuro-circuitry based signature generator 112 may be configured to generate a particular type of the neuro-circuitry based signature. In other embodiments, neuro-circuitry based signature generator 112 may receive one or more controlling inputs indicating specific models within neurocircuitry database 116 to combine into a neuro-circuitry based signature. These controlling inputs may be specified by a user or generated in any other suitable way. Neuro-circuitry based signature generator 112 may retrieve information about one or more neurocircuit models from neurocircuitry database(s) 116 based on one or more input parameters, including user input indicative of characteristics of the clinical trial, a compound being evaluated, and a condition or disease state of a subject. Based on one or more inputs, neuro-circuitry based signature generator 112 may select a set of neurocircuit models to apply to measurement data 104 to determine a neuro-circuitry based signature.

Alternatively or additionally, controlling inputs to neuro-circuitry based signature generator 112 may specify the manner in which model outputs calculated in accordance with multiple neurocircuitry models may be combined into a neuro-circuitry based signature. These, and any other suitable inputs that specify which neurocircuit models to apply to the measurement data 104, may be provided in any suitable way. In some embodiments, for example, the inputs may be provided through a user interface, which may be implemented using command line or a graphical user interface programming constructs. Alternatively or additionally, the inputs that specify which neurocircuits are used and how the model outputs produced by each are combined into a neuro-circuitry based signature may be programmed in a scripting language or other programming language. In such an embodiment, a script or other program file may be stored and executed by neuro-circuitry based signature generator 112.

Regardless of how information controlling neuro-circuitry based signature generator 112 is specified, operation in accordance with the specified control information may cause neuro-circuitry based signature generator 112 to retrieve information stored in neurocircuitry database(s) 116 defining neurocircuit models and analyze the measurement data in accordance with the one or more neurocircuit models to generate a neuro-circuitry based signature. Execution of a neurocircuit model may generate a model output based on classifying the measurement data in accordance with a neurocircuit. The model output may be a value along a spectrum of model outputs that captures a degree of behavior or cognition. A neuro-circuitry based signature generated by neuro-circuitry based signature generator 112 may be based on or include the model output as part of the neuro-circuitry based signature. Multiple neurocircuit models may classify the measurement data, and the neuro-circuitry based signature may include or be based on model outputs corresponding to the different neurocircuit models.

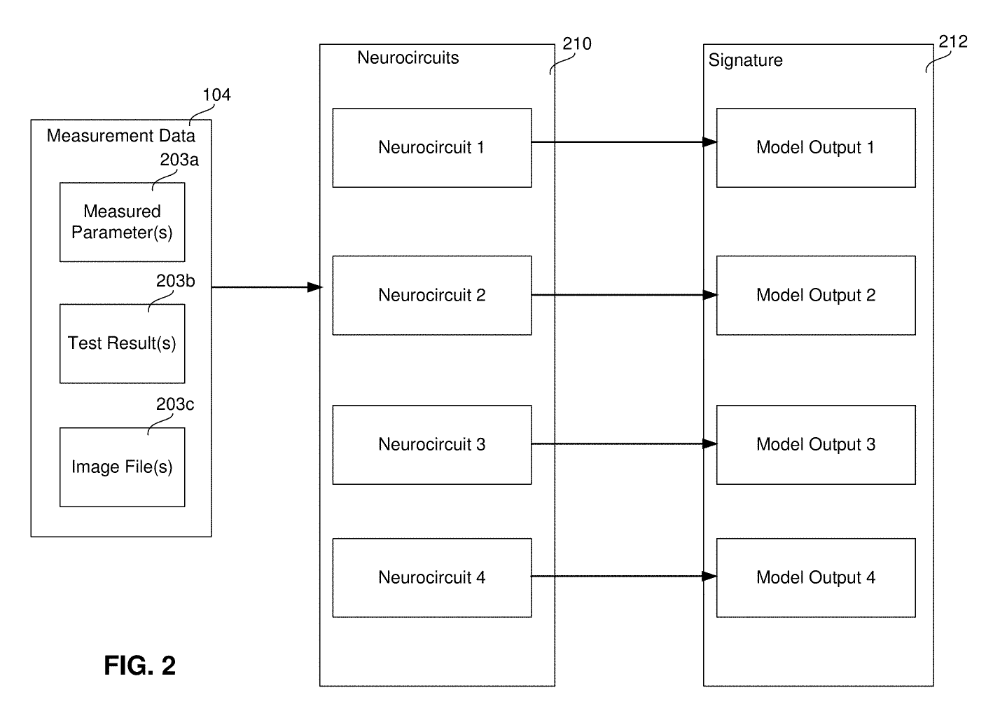

FIG. 2 illustrates an exemplary schematic of data flow in a process used to determine a neuro-circuitry based signature by neuro-circuitry based signature generator 112. Measurement data 104 may include data acquired using different measurement methods and techniques, as discussed above. As shown in FIG. 2, measurement data 104 may include measured parameter(s) 203a, test result(s) 203b, and/or image file(s) 203c. Multiple types of measurement data, and multiple values of each type, may be available, reflecting multiple measurements made on a subject or group of subjects.

Inputs into neurocircuits 210 may include measurement data 104. Outputs from neurocircuit models 210 include model outputs, which form neuro-circuitry based signature 212. Any suitable number of neurocircuit models may be used in determining a neuro-circuitry based signature. As depicted in FIG. 2, neurocircuit models 210 include neurocircuit 1, neurocircuit 2, neurocircuit 3, and neurocircuit 4. In the example of a neuro-circuitry based signature generated as part of a trial to assess the effectiveness of an investigational compound in improving focus, these neurocircuits may represent focus, cognition, and other neurocircuits known to show activity correlated with focus. However, it should be appreciated that four neurocircuit models are shown for an example only and more or fewer models may be used in some embodiments.

Multiple model outputs from different neurocircuits may generate a neuro-circuitry based signature. As shown in FIG. 2, neuro-circuitry based signature 212 includes model output 1, model output 2, model output 3, and model output 4, each generated by one neurocircuit model. However, the mapping of neurocircuits to model outputs does not necessarily have to be a one-to-one mapping. In some embodiments, a neurocircuit may output more than one model output or multiple values characterizing the model output. In some embodiments, a model output may depend on multiple neurocircuits.

As an example, images acquired using functional imaging techniques (e.g., fMRI) may indicate activity level for different regions of a patient's brain. Those activity levels and brain regions may be provided as inputs to one or more neurocircuit models, which analyze the activity levels and brain regions to generate one or more model outputs.

The models may specify one or more computations to generate a model output from measurement data. A single data value might, in some embodiments, be compared to an average value and the difference scaled to produce a model output. In some models, multiple measurement data values might be processed, such as to determine an average or other statistics of multiple measurements. The distribution of those data values might be compared to statistics for similar measurements on healthy individuals and the difference scaled, linearly or nonlinearly, to produce a model output. However, it should be appreciated that any suitable computation or computations may be specified by a model, and the specific computations specified by any model may depend on the type of measurement data and the brain function being modeled.

The collection of model outputs may form a neuro-circuitry based signature. That neuro-circuitry based signature may characterize a subject from which the measurement data was collected, and may, for example, be representative of a condition of the subject. In some embodiments, the neuro-circuitry based signature may contain model outputs revealing a current state of the subject. In other embodiments, the model outputs in the neuro-circuitry based signature may characterize a change in model outputs between two states. In that case, the neuro-circuitry based signature may alternatively or additionally characterize a change in the state of the subject, such as a change that may occur when the subject is administered a drug. In that case, the neuro-circuitry based signature may also characterize a response to the drug, such that the neuro-circuitry based signature alternatively or additionally may be used as the neuro-circuitry based signature of the drug or other intervention administered to the patient.

In yet other embodiments, measurement data supplied to a model may characterize a population of subjects. In that case, the neuro-circuitry based signature may characterize the population or the response of the population to a drug or other intervention. Accordingly, in each instance in which a neuro-circuitry based signature of a single subject is described, it should be appreciated that a neuro-circuitry based signature or a population alternatively or additionally could be computed. Likewise, in each instance in which a neuro-circuitry based signature is described as characterizing a subject at a particular time, it should be appreciated that a neuro-circuitry based signature characterizing a change in the subject between two states may alternatively or additionally be computed. Moreover, it should be appreciated that a placebo may have a powerful influence on a variety of behavioral and neurocircuitry based measures, and “treatment” with a placebo may be a state. The comparison of placebo versus active drug therapy may be considered in the signature generator. Placebo data on the same patient or volunteer population can be pooled across multiple studies enhancing the power and sensitivity of the analyses performed.

A neuro-circuitry based signature, in some embodiments, may also contain information in addition to the model outputs. The neuro-circuitry based signature may contain information indicating conditions under which measurements were made and/or interventions on the subject between the times at which measurements used to derive model outputs were made. As another example, the neuro-circuitry based signature may include identifying data for the subject (which may be a code or other mechanism that allows comparison of the same subject over time without personally identifying the subject), which may be personally identifying information or anonymized, but sufficient to allow relating or comparing neuro-circuitry based signatures for the same subject. As yet a further example, a signature may include genomics and pharmacokinetic and pharmacodynamic modeling data.

Returning to FIG. 1, once the neuro-circuitry based signature is generated it may be analyzed, such as by comparison to other neuro-circuitry based signatures. In some embodiments, neuro-circuitry based signature analyzer 114 may analyze a patient's neuro-circuitry based signature by comparing the neuro-circuitry based signature to one or more reference neuro-circuitry based signatures to generate an indication result 120. The comparison of the neuro-circuitry based signature to a reference neuro-circuitry based signature may indicate the patient's condition, indicate an evaluation of a compound administered to the patient, and/or provide an aspect of a clinical trial design. The type of result generated may depend on the type of reference neuro-circuitry based signatures used by neuro-circuitry based signature analyzer 114 to generate the result. The specific neuro-circuitry based signature used as a reference and the specific type of result produced may depend on the analysis the neuro-circuitry based signature analyzer 114 is configured to perform. In some embodiments, that configuration may be established when neuro-circuitry based signature analyzer 114 is constructed. For example, a neuro-circuitry based signature analyzer may be implemented as a set of computer-executable instructions loaded for execution on a processor. Those instructions may identify predefined reference neuro-circuitry based signatures and desired results. Alternatively or additionally, the configuration of neuro-circuitry based signature analyzer 114 may be established by user input. As with neuro-circuitry based signature generator 112, the controlling input to neuro-circuitry based signature analyzer 114 may be in the form of commands, entered through a command line or graphical user interface, a scripting file or in any other suitable format. In that case, neuro-circuitry based signature analyzer 114 may be configured to access a reference neuro-circuitry based signature specified in the input and produce a corresponding result.

Neuro-circuitry based signature analyzer 114 may access a reference neuro-circuitry based signature from reference neuro-circuitry based signature database(s) 118. A reference neuro-circuitry based signature may include one or more model outputs corresponding to one of more neurocircuits, which may be computed using the same or similar neurocircuit models in neuro-circuitry based signature generator 112 to produce a neuro-circuitry based signature using measurements collected during a trial. The reference neuro-circuitry based signature may comprise a vector of model outputs where each model output in the vector corresponds to a value along a spectrum of values for a certain neurocircuit. In this manner, the vector of model outputs for the reference neuro-circuitry based signature may represent outputs from multiple neurocircuit models. Any suitable format may be used to structure a reference neuro-circuitry based signature database, but in some embodiments a reference neuro-circuitry based signature database may have a table format configured to store the vectors corresponding to multiple reference neuro-circuitry based signatures. As with the generated neuro-circuitry based signature, the reference neuro-circuitry based signatures may also include information indicating the conditions under which the neuro-circuitry based signature was generated. This information may be used to select an appropriate reference neuro-circuitry based signature for analysis and/or may be used in subsequent analysis.

One or more types of reference neuro-circuitry based signatures may be stored in reference neuro-circuitry based signature database(s) 118. Reference neuro-circuitry based signatures may include, for example, neuro-circuitry based signatures derived from people with a certain condition or a condition of a certain type. Such a reference neuro-circuitry based signature may be obtained by acquiring measurement data from people identified as having a particular type of condition and generating a neuro-circuitry based signature based on the measurement data. In such an embodiment, a reference neuro-circuitry based signature may be generated with one or more neurocircuit models corresponding to brain functions that the condition may impact.

Accordingly, decision support tool 110 may be configured to generate or analyze neuro-circuitry based signatures using a subset of available model outputs, with the specific model outputs used depending on one or more parameters. Those parameters may include the condition or type of condition that a subject has or is suspected of having, the condition or type of condition that a drug or other intervention is effective against, or is theorized to be effective against, or the mechanism of action, or theorized mechanism of action, of a drug with which a subject is treated.

In some embodiments, reference neuro-circuitry based signature database 118 may include sets of model outputs assembled into neuro-circuitry based signatures. In other embodiments, reference neuro-circuitry based signature database 118 may store information defining reference neuro-circuitry based signatures in other formats. For example, it may store model outputs and associated information from which those model outputs may be selected and assembled into a neuro-circuitry based signature. The associated information may identify, for example, a subject or subject population, a condition or type of condition assigned to the subject, a state of the subject in which the measurements from which the model output was computed were collected, a drug or other intervention provided to the subject prior to making the measurement. Other results output by the tool may include protocol optimization recommendations, such as an adaptive design for the trial.

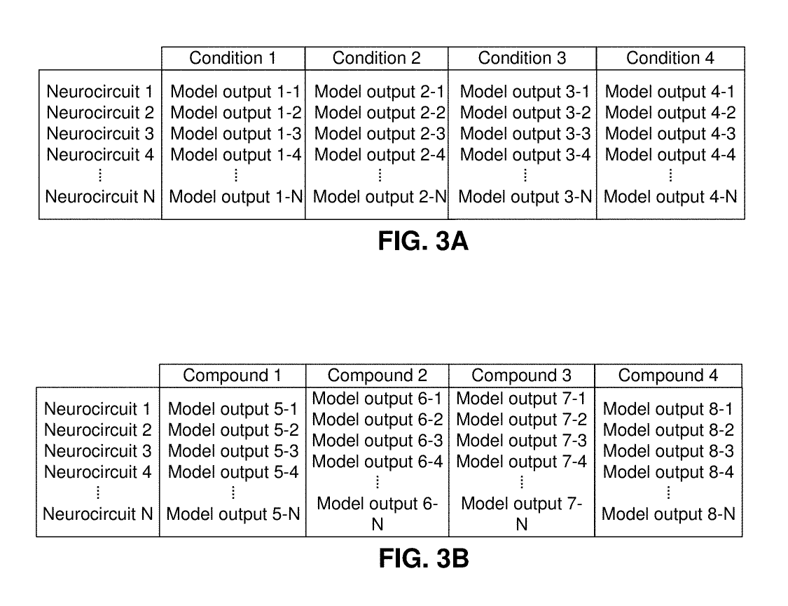

FIG. 3A schematically illustrates an exemplary database of reference neuro-circuitry based signatures for different conditions. Each column in the database corresponds to a different condition where each row in the database corresponds to a different neurocircuit. Here, “Condition 1” has a reference neuro-circuitry based signature having “Model output 1-1,” “Model output 1-2,” “Model output 1-3,” “Model output 1-4,” . . . “Model output 1-N,” where N is the number of neurocircuits represented in the database. The model outputs in this example represent model outputs computed using a neurocircuitry model for a corresponding neurocircuit. In this manner, each column in the database depicted in FIG. 3A corresponds to a different reference neuro-circuitry based signature. It should be appreciated that other database formats suitable for storing reference neuro-circuitry based signatures may be used.

FIG. 3B shows an exemplary database of reference neuro-circuitry based signatures for different compounds. Each column in the database corresponds to a different compound where each row in the database corresponds to a different neurocircuit. Here, “Compound 1” has a reference neuro-circuitry based signature having “Model output 5-1,” “Model output 5-2,” “Model output 5-3,” “Model output 5-4,” . . . “Model output 5-N,” where N is the number of neurocircuits represented in the database. The model outputs in this example represent model outputs computed using a neurocircuitry model for a corresponding neurocircuit. In this manner, each column in the database shown in FIG. 3B corresponds to a reference neuro-circuitry based signature for a different compound.

In the examples of FIGS. 3A and 3B, each neuro-circuitry based signature may be for a single subject or aggregated across multiple subjects. Likewise, each database may contain neuro-circuitry based signatures associated with a homogeneous or heterogeneous population. If heterogeneous, the database may alternatively include, associated with each neuro-circuitry based signature, information from which data associated with specific groups may be selected. In that scenario, a neuro-circuitry based signature for that specific group may be assembled from the data by executing search or filter commands in a computer providing access to the database. Moreover, it should be appreciated that FIGS. 3A and 3B are examples of reference neuro-circuitry based signatures as reference neuro-circuitry based signatures may alternatively or additionally be in any other form as described herein or in any other suitable form that enables a comparison to one or more other neuro-circuitry based signatures.

Regardless of how data on reference neuro-circuitry based signatures is stored and accessed, neuro-circuitry based signature analyzer 114 may compare a neuro-circuitry based signature of a patient to one or more reference neuro-circuitry based signatures from reference database(s) 118. By comparing the neuro-circuitry based signature and a reference neuro-circuitry based signature, an evaluation of the similarity between the neuro-circuitry based signature and the reference neuro-circuitry based signature may be determined. Comparison between a neuro-circuitry based signature and a reference neuro-circuitry based signature may be performed by comparing model outputs that correspond to the same neurocircuit in the neuro-circuitry based signature and the reference neuro-circuitry based signature. In some embodiments, the comparison may result in a computed value representative of a degree of similarity between the neuro-circuitry based signatures being compared. In some embodiments, comparison of a neuro-circuitry based signature to a reference neuro-circuitry based signature may include applying weighting criteria to different model outputs such that model outputs for some neurocircuits factor more into determining a level of similarity between a neuro-circuitry based signature and a reference neuro-circuitry based signature than other neurocircuits. The weighting criteria applied to model outputs may apply higher weighting to model outputs for neurocircuits that have been shown to be affected more than other neurocircuits when a condition is active in a subject.

Accordingly, the comparison of neuro-circuitry based signatures may be made as a weighted and/or non-linear combination of comparisons for like model outputs included in the neuro-circuitry based signatures being compared. Individual comparisons may be made based on model outputs derived from the same neurocircuit model or for models that are representative of the same or similar neurocircuit. Differences in model outputs that are tightly correlated with a behavioral condition, for example, may be given higher weighting in comparing neuro-circuitry based signatures than those that are only weakly correlated.

In some embodiments, the comparison may be numeric, with the comparison being represented as an absolute or relative difference. In other embodiments, model outputs may be compared by determining whether the model output is the same in both the neuro-circuitry based signature and the reference neuro-circuitry based signature for the same neurocircuit, such as by using Boolean logic techniques. In making a Boolean comparison, values that differ by less than a threshold amount may be treated as being the same. Neuro-circuitry based signatures may also be compared by using fuzzy logic techniques. In some embodiments, comparison of model outputs may include determining a level of similarity between a model output in the neuro-circuitry based signature and the reference neuro-circuitry based signature that correspond to the same neurocircuit. A threshold level may be applied in the comparison between a neuro-circuitry based signature and a reference neuro-circuitry based signature to provide a threshold degree of similarity, which may be reflected in the weighting of the individual model outputs. Differences that are within a statistical measure of variation indicating normal variation may be given little or no weight, whereas variations that are statistically significant may be given much greater weight.

As shown in FIG. 1, neuro-circuitry based signature analyzer 114 may generate an indication result 120 based on the comparison between a subject's neuro-circuitry based signature and one or more reference neuro-circuitry based signatures. The result may provide information about the level of similarity between the subject's neuro-circuitry based signature and one or more reference neuro-circuitry based signatures. The nature of the information may depend on the nature of the reference neuro-circuitry based signature used in the comparison as well as the conditions under which measurement was made on the subject. Accordingly, FIG. 1 illustrates that decision support tool 110 may be operated, in different modes, to produce any one or more of multiple types of results. Indication result 120 may include, for example, information related to a CNS condition, an evaluation of a compound, and/or an aspect of clinical trial design.

For example, by comparing a patient's neuro-circuitry based signature to reference neuro-circuitry based signatures corresponding to different conditions, such as the reference neuro-circuitry based signatures of FIG. 3A, neuro-circuitry based signature analyzer 114 may produce a result of whether the patient's neuro-circuitry based signature is similar to a reference neuro-circuitry based signature for a condition. In such instances, the indication result 120 may be formatted as a diagnosis for the patient, which may be arrived at by identifying a condition represented by a reference neuro-circuitry based signature that has a high level of similarity with the patient's neuro-circuitry based signature. In some embodiments, the result may include multiple conditions where the conditions are ranked by the level of similarity based on the comparison between the patient's neuro-circuitry based signature to the reference neuro-circuitry based signatures corresponding to the multiple conditions.

As another example, a comparison between the subject's neuro-circuitry based signature and a reference neuro-circuitry based signature associated with a reference compound may provide an indication result 120 indicating whether the subject is experiencing brain functions similar to a subject who has taken the reference compound.

In some embodiments, generating the result includes generating a result of whether the compound alleviates one or more symptoms associated with the CNS condition. In this case, a symptom may be assessed based on the measurement data and represented in the patient's neuro-circuitry based signature. A comparison between the neuro-circuitry based signature and a reference neuro-circuitry based signature may indicate whether the symptom has been alleviated.

Such results may be used in any of multiple ways. In scenarios in which the efficacy of the reference compounds in treating one or more CNS conditions is known, a comparison indicating a high degree of similarity to a reference compound may indicate the utility of an investigational drug. In this manner, an investigational drug may be evaluated by comparing its effects on a person's brain functions to known effects of previously studied drugs. The investigational drug may be identified as being likely effective in treating the same condition as a known drug.

In other scenarios, a decision support tool 110 may provide information used in designing clinical trials for an investigational drug. A researcher using the decision support tool 110, for example, may have a theory about CNS conditions that can be effectively treated by an investigational drug. In establishing a clinical trial, or even in selecting preclinical testing, that researcher may input the CNS condition as part of a command requesting the tool indicate measurements to be performed during a trial. In response, decision support tool 110 may access reference neuro-circuitry based signature database 118 to select neuro-circuitry based signatures of known drugs based on their effectiveness in treating the input CNS condition.

Neuro-circuitry based signature analyzer 114 may analyze the selected neuro-circuitry based signatures to identify characteristics that distinguish the effective from the ineffective drugs. Analyzer 114 may relate these characteristics to measurements that will collect information on the characteristic for an investigational drug. In the implementation illustrated in FIG. 1, neuro-circuitry based signature analyzer 114 may identify model outputs that distinguish effective from ineffective drugs. Because each model output is associated with that neurocircuit model, neuro-circuitry based signature analyzer 114 may identify neurocircuit models useful in evaluating the investigational drug to quickly make an assessment of whether that investigational drug is effective against the theorized CNS condition. The neurocircuit models are themselves associated with measurements, allowing neuro-circuitry based signature analyzer 114 to identify measurements to be performed on the investigational drug. These identified measurements may be a portion of the indicated clinical trial design.

In some embodiments, decision support tool 110 may aid a researcher in identifying a theorized condition against which an investigational drug will be effective. Identification may be performed with a relatively small amount of data, such as might be collected at the preclinical or phase one stage of a clinical trial process. From this small amount of data, decision support tool 110 may guide what condition or conditions an investigational drug is tested against by identifying a target condition or conditions that the investigational drug is likely to be effective against. Identification of a target condition may impact the design of subsequent steps in a clinical trial process in any one or more ways.

In some scenarios, identifying likely effectiveness against a CNS condition may lead to designing a trial to test whether an investigational drug is effective against that condition, which in turn may lead to the tool indicating elements of the clinical trial protocol. Designing a clinical trial may include identifying patients diagnosed with the one or more conditions and determining measurement techniques for evaluation of the one or more conditions.

In some embodiments, reference neuro-circuitry based signatures may have more information associated with them than simply what drug was administered to subjects from which the reference neuro-circuitry based signature was generated. Associated information may include, for example, dosing levels, duration of treatment, or other parameters of administration. By selecting reference neuro-circuitry based signatures that indicate effectiveness, the associated dosing levels, duration of treatment or other parameters of administration that provided effectiveness may likewise be determined. The values of these parameters, indicating effectiveness, may be used to define treatment in one or more arms of a clinical trial.

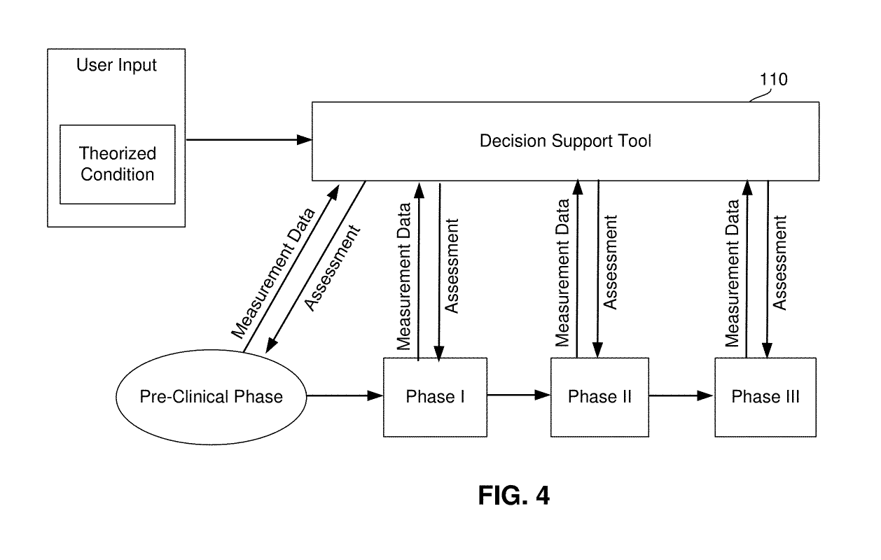

FIG. 4 illustrates an exemplary schematic of phases in clinical research, which includes phases to evaluate the safety, efficacy, and/or effectiveness of drugs as treatments. Evaluation of a compound's safety may include determining a dosing level or dose range that is toxic to a subject, including non-human and human subjects. A compound's efficacy can be assessed by determining whether the compound is able to influence an outcome of interest in one or more individuals. The compound may be evaluated for its effectiveness in influencing one or more diseases. One or more clinical trials may be developed for a particular phase illustrated in FIG. 4 using the techniques described herein. The decision support tool 110 may be implemented at one or more clinical trials at different phases in clinical research. The decision support tool 110 may receive user input, such as a theorized condition that against which an investigational drug will be effective as shown in FIG. 4, which can be used in collecting measurement data and generating an assessment at one or more phases of clinical research. However, it should be appreciated that any suitable user input may be provided, including clinical diagnoses, symptom specific targets, and research domain criteria. Measurement data collected at a phase may be provided to the decision support tool 110, which may generate an assessment based on the measurement data. The assessment may be used to evaluate the drug at that phase in clinical research and/or determine the design on a clinical trial in a subsequent phase.

The pre-clinical phase includes identifying a compound as a drug candidate, which may include evaluating the efficacy and/or toxicity of the compound on a non-human subject, including in vitro (e.g., cell culture) and in vivo (e.g., animal) experiments.

After the pre-clinical phase, phase I of a clinical trial may include testing of the compound on humans to evaluate whether the compound is safe for further testing. Phase I may include evaluating the effect of different doses of the compound on a group of people. An outcome of phase I may include identifying effective and safe dose levels, such as a dose that works best and/or a dose that is least harmful. A clinical trial in phase I may be designed to demonstrate the compound as being safe to administer to human beings. Clinicians may collect data on the dose level, timing of administering the compound, method of administering the compound (e.g., orally, through a vein) and safety of the treatments.

Evaluation of the compound may proceed in a clinical trial to Phase II where the compound can be assessed on patients to further determine its efficacy and safety. Phase II may include evaluating whether the compound has a biological activity or effect. A clinical trial in phase II may be designed to evaluate how well the compound works in treating a particular disease and the safety of the compound.

Further evaluation of the compound may proceed in phase III, which may include assessing the compound's effectiveness on one or more diseases. Evaluation during phase III may include comparing the influence of the compound in treating a disease with a standard treatment for the disease. A clinical trial in phase III may evaluate the effectiveness of the compound in treating a particular disease by administering the compound to a group of individuals diagnosed with the disease and/or symptoms indicative of the disease.

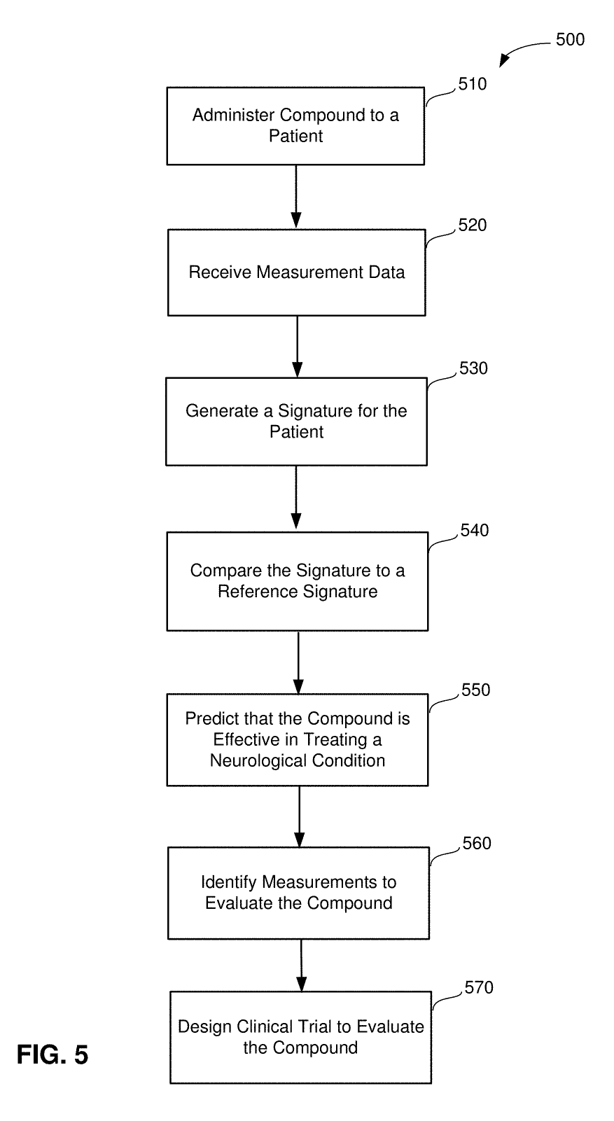

Decision support tool 110 may be used to alter a conventional flow of testing of investigational drugs being developed for treatment of CNS conditions. The tool may provide information, based on measurements made at one phase, which guides how a subsequent phase is performed. FIG. 5 depicts an exemplary method for designing a clinical trial to evaluate a compound, which may be implemented using a decision support tool, such as decision support tool 100.

Method 500 begins at step 510 where a compound is administered to a patient. The patient may be identified as healthy, such as in a phase I clinical trial. Measurement data is collected on the patient and received by the decision support tool at step 520. At step 530, the measurement data may be used to generate a neuro-circuitry based signature for the patient using one or more neurocircuit models, which may be performed by neuro-circuitry based signature generator 112. At step 540, the decision support tool may compare the neuro-circuitry based signature to one or more reference neuro-circuitry based signatures, which may be performed by neuro-circuitry based signature analyzer 114. The comparison may include comparing the neuro-circuitry based signature to reference neuro-circuitry based signatures that correspond to different compounds, as discussed above. At step 550, the tool may predict, based on the comparison, that the compound is effective in treating a CNS condition as a result. Such a result may be generated, for example, by neuro-circuitry based signature analyzer 114.

At step 560, identification of measurements to evaluate the compound may be determined. Measurement techniques may depend on the CNS condition the compound is predicted to treat. The measurements identified may provide a level of accuracy in assessing and evaluating changes in the predicted CNS condition. As described herein, that identification may be based on an analysis of a database that contains model outputs for multiple neurocircuits for patients with a condition that was alleviated by treatment.

At step 570, a clinical trial to evaluate the compound may be evaluated. Design of a clinical trial to evaluate the compound may include designing a phase II and/or phase III clinical trial. A protocol for the clinical trial may include identifying subjects for the clinical trial diagnosed with the predicted CNS condition and/or performing measurements on the subjects, such as the measurements identified in step 560 of method 500. Enrollment of subjects into the clinical trial may include identifying one or more symptoms associated with the predicted CNS condition, which also may be performed using neuro-circuitry based signature analysis—in this case by comparing neuro-circuitry based signatures of subjects to neuro-circuitry based signatures of subjects diagnosed with the condition. Equipment for performing the identified measurements may be acquired at sites of the clinical trial for evaluation of the predicted CNS condition.

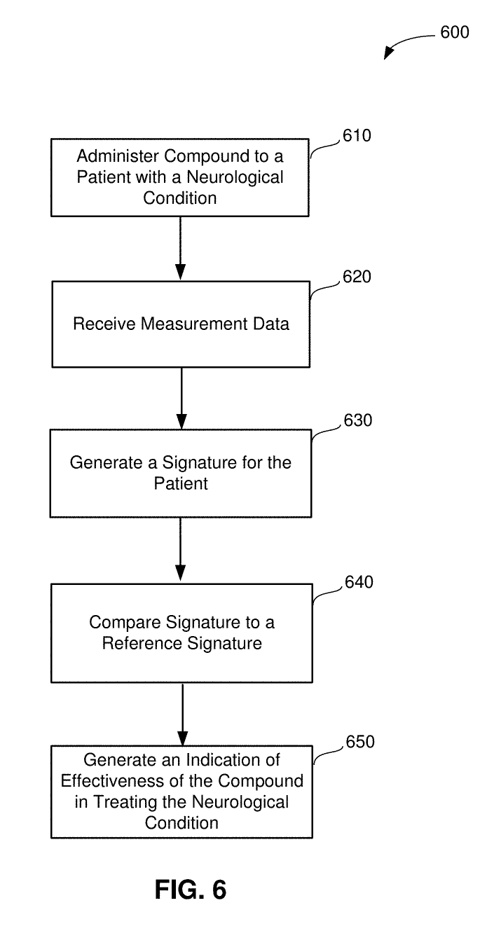

FIG. 6 depicts an exemplary method for determining the effectiveness of a compound in treating a CNS condition, which may be performed by decision support tool 110 (FIG. 1). Method 600 begins at step 610 where a compound is administered to a patient diagnosed with a CNS condition, such as in a phase II or III clinical trial. In some embodiments, the compound may have been previously evaluated in a phase I clinical trial on healthy subjects as having a particular biological activity or effect associated with the CNS condition. Although FIG. 6 illustrates measurements performed on subjects diagnosed with a condition, the processing at step 610 may be performed on subjects without a diagnosis, such as may occur in Phase I of a trial.

Measurement data is obtained for a patient and received by the decision support tool by step 620. At step 630, the measurement data may be used to generate a neuro-circuitry based signature for the patient using one or more neurocircuit models, which may be performed by neuro-circuitry based signature generator 112. As discussed above, the neuro-circuitry based signature may include model outputs associated with different neurocircuits. At step 640, the decision support tool may compare the neuro-circuitry based signature to one or more reference neuro-circuitry based signatures, which may be performed by neuro-circuitry based signature analyzer 114. At step 650, an indication of effectiveness of the compound for treating the CNS condition may be generated, such as by neuro-circuitry based signature analyzer 114.

In some embodiments, a computed result indicating the effectiveness of the compound in treating the CNS condition may be used to evaluate or modify the clinical trial design. If the result indicates that the compound is likely effective in treating the CNS condition at step 660, then the clinical trial may proceed to be conducted at step 670. If deemed effective, the information generated by the tool may form a dose and/or exposure strategy or recommendation for conducting the trial. However, if the compound is determined to be not effective in treating the CNS condition at step 660, then an alternative clinical trial may be designed. In some embodiments, another CNS condition may be identified by a decision support tool at step 680. In such embodiments, the design of the clinical trial may be changed at step 690 to evaluate the condition identified at step 680. Changing the design of a clinical trial may include changing one or more aspects of the clinical trial that relate to evaluating the effectiveness of the compound on the condition identified at step 680. Changing design of a clinical trial to evaluate the effectiveness of the drug for the condition identified in step 680 may include changing the types of measurements to perform as part of the conducting the clinical trial such that the condition identified at step 680 is evaluated, changing the which neurocircuits are used in generating a neuro-circuitry based signature, and changing selection criteria of patients to be admitted as subjects in the trial. Conducting a clinical trial for a compound may include The changed clinical trial may then be conducted at step 690.

Processing as described herein may be performed using any suitable hardware and/or software. Measurement data 104, for example, may be generated by one or more types of imaging or laboratory machines. Computing equipment may be configured to generate reference neuro-circuitry based signatures, generate investigational neuro-circuitry based signatures and compare neuro-circuitry based signatures. Likewise, computer databases may be specifically organized to store neurocircuitry models and reference neuro-circuitry based signatures in a way that they can be accessed to generate and analyze neuro-circuitry based signatures as described herein. Computing equipment may also be configured to receive inputs, such a theorized condition that an investigational drug can treat and determine protocol elements of a clinical trial, as described above.

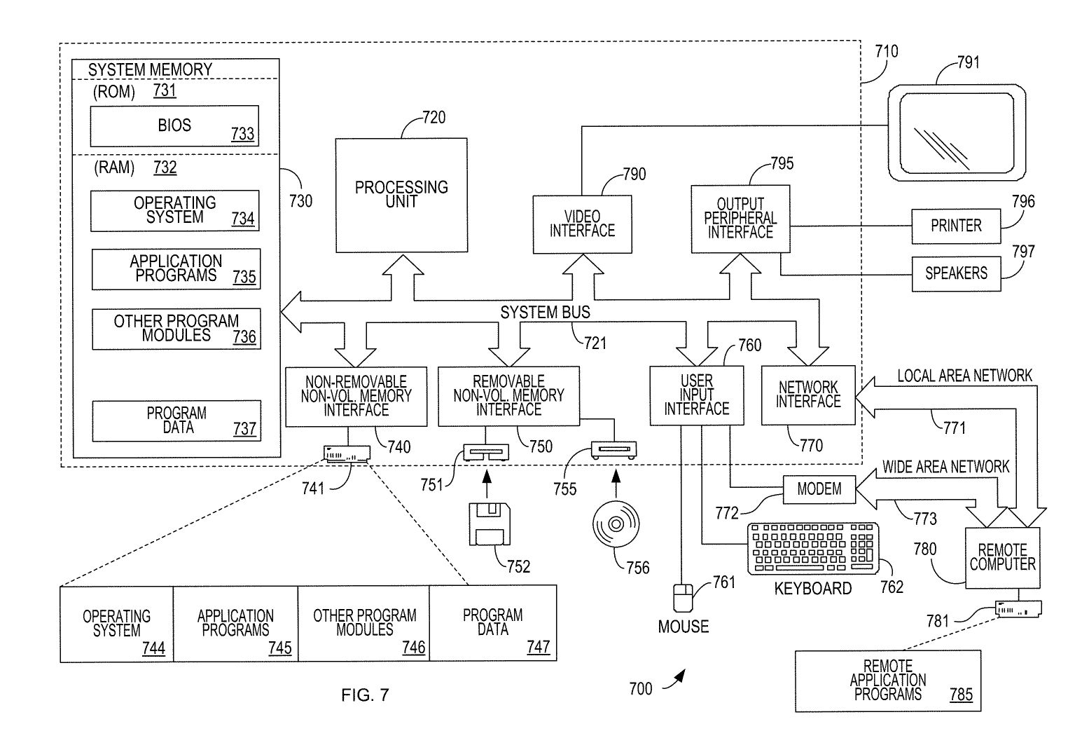

FIG. 7 illustrates an example of a suitable computing system environment 700 on which the invention may be implemented. The computing system environment 700 is only one example of a suitable computing environment and is not intended to suggest any limitation as to the scope of use or functionality of the invention. Neither should the computing environment 700 be interpreted as having any dependency or requirement relating to any one or combination of components illustrated in the exemplary operating environment 700.

The invention is operational with numerous other general purpose or special purpose computing system environments or configurations. Examples of well-known computing systems, environments, and/or configurations that may be suitable for use with the invention include, but are not limited to, personal computers, server computers, smartphones, tablets, hand-held or laptop devices, cloud computers, multiprocessor systems, microprocessor-based systems, network PCs, minicomputers, mainframe computers, distributed computing environments that include any of the above systems or devices, and the like. Some of the elements illustrated in FIG. 7 may not be present, depending on the specific type of computing device. Alternatively, additional elements may be present in some implementations.

The computing environment may execute computer-executable instructions, such as program modules. Generally, program modules include routines, programs, objects, components, data structures, etc. that perform particular tasks or implement particular abstract data types. Some embodiments may also be practiced in distributed computing environments where tasks are performed by remote processing devices that are linked through a communications network. These distributed systems may be what are known as enterprise computing systems or, in some embodiments, may be “cloud” computing systems. In a distributed computing environment, program modules may be located in both local and/or remote computer storage media including memory storage devices.

With reference to FIG. 7, an exemplary system for implementing the invention includes a general purpose computing device in the form of a computer 710. Components of computer 710 may include, but are not limited to, a processing unit 720, a system memory 730, and a system bus 721 that couples various system components including the system memory to the processing unit 720. The system bus 721 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

Computer 710 typically includes a variety of computer readable media. Computer readable media can be any available media that can be accessed by computer 710 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 710.

Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 730 includes computer storage media in the form of volatile and/or nonvolatile memory such as read only memory (ROM) 731 and random access memory (RAM) 732. A basic input/output system 733 (BIOS), containing the basic routines that help to transfer information between elements within computer 710, such as during start-up, may be stored in ROM 731. RAM 732 may contain data and/or program modules that are immediately accessible to and/or presently being operated on by processing unit 720. By way of example, and not limitation, FIG. 7 illustrates operating system 734, application programs 735, other program modules 736, and program data 737.

The computer 710 may also include other removable/non-removable, volatile/nonvolatile computer storage media. By way of example only, FIG. 7 illustrates a hard disk drive 741 that reads from or writes to non-removable, nonvolatile magnetic media. Such a hard disk drive may be implemented by a rotating disk drive or as a solid state drive, such as is implemented with FLASH memory.

FIG. 7 also illustrates a magnetic disk drive 751 that reads from or writes to a removable, nonvolatile magnetic disk 752, and an optical disk drive 755 that reads from or writes to a removable, nonvolatile optical disk 756 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 741 may be connected to the system bus 721 through a non-removable memory interface such as interface 740, and magnetic disk drive 751 and optical disk drive 755 may be connected to the system bus 721 by a removable memory interface, such as interface 750. However, it should be appreciated that, in some embodiments, some or all of the computer readable media available to a device may be accessed over a communication network.

The drives and their associated computer storage media discussed above and illustrated in FIG. 7, provide storage of computer readable instructions, data structures, program modules and other data for the computer 710. In FIG. 7, for example, hard disk drive 741 is illustrated as storing operating system 744, application programs 745, other program modules 746, and program data 747. Note that these components can either be the same as or different from operating system 734, application programs 735, other program modules 736, and program data 737. Operating system 744, application programs 745, other program modules 746, and program data 747 are given different numbers here to illustrate that, at a minimum, they are different copies.

A computing environment may include one or more input/output devices. Some such input/out devices may provide a user interface. A user may enter commands and information into the computer 710 through input devices such as a keyboard 762 and pointing device 761, depicted as a mouse. However, other forms of pointing devices may be used, including a trackball, touch pad or touch screen. Other input devices (not shown) may include a microphone, joystick, game pad, satellite dish, scanner, or the like. The microphone, for example, may support voice input, which may be recorded as an audio file or may be translated, such as using speech recognition, to a text format for further processing. These and other input devices are often connected to the processing unit 720 through a user input interface 760 that is coupled to the system bus, but may be connected by other interface and bus structures, such as a parallel port, game port or a universal serial bus (USB).

The computing device may include one or more output devices, including an output device that may form a portion of a user interface. A monitor 791 or other type of display device may also connect to the system bus 721 via an interface, such as a video interface 790, to form a visual output device. In addition to the monitor, computers may also include other peripheral output devices such as speakers 797 and printer 796, which may be connected through an output peripheral interface 795. The speaker, for example, may enable output via synthesized voice or in any other suitable way.

The computer 710 may operate in a networked environment using logical connections to one or more remote computers, such as a remote computer 780. The remote computer 780 may be a personal computer, a server, a router, a network PC, a peer device or other common network node, and typically includes many or all of the elements described above relative to the computer 710, although only a memory storage device 781 has been illustrated in FIG. 7. The logical connections depicted in FIG. 7 include a local area network (LAN) 771 and a wide area network (WAN) 773, but may also include other networks. Such networking environments are commonplace in offices, enterprise-wide computer networks, intranets and the Internet. Alternatively or additionally, the WAN may include a cellular network.

When used in a LAN networking environment, the computer 710 is connected to the LAN 771 through a network interface or adapter 770. When used in a WAN networking environment, the computer 710 typically includes a modem 772 or other means for establishing communications over the WAN 773, such as the Internet. The modem 772, which may be internal or external, may be connected to the system bus 721 via the user input interface 760, or other appropriate mechanism.

In a networked environment, program modules depicted relative to the computer 710, or portions thereof, may be stored in the remote memory storage device. By way of example, and not limitation, FIG. 7 illustrates remote application programs 785 as residing on memory device 781. It will be appreciated that the network connections shown are exemplary and other means of establishing a communications link between the computers may be used.

Depending on the nature of the computing device, one or more additional elements may be present. For example, a smart phone or other portable electronic device may include a camera, capable of capturing still or video images. In some embodiments, a computing device may include sensors such as a global positioning system (GPS) to sense location and inertial sensors such as a compass, an inclinometer and/o ran accelerometer. The operating system may include utilities to control these devices to capture data from them and make it available to applications executing on the computing device.

As another example, in some embodiments, a computing device may include a network interface to implement a personal area network. Such an interface may operate in accordance with any suitable technology, including a Bluetooth, Zigbee or an 802.11 ad hoc mode, for example.

Having thus described several aspects of at least one embodiment of this invention, it is to be appreciated that various alterations, modifications, and improvements will readily occur to those skilled in the art.

For example, it is described that neuro-circuitry based signatures are computed based on measurements made on a subject in a particular state. In some embodiments, all of the measurements will be made at the same time, such as during a single visit to a health care center or during a single day. However, it should be appreciated that the equipment to make measurements may be distributed at many locations, such that a subject cannot practically be given all measurements in a single day. Accordingly, it should be understood that measurements made while the patient is in the same condition, such as before receiving any treatment or within 4 hours of receiving an administration of a drug, may be considered to represent the same state of the subject, even though those measurements are made on different days or in different locations.

Further, neuro-circuitry based signature generator 112 was described, in some embodiments, as generating a neuro-circuitry based signature representative of a change in a subject between two states and, in some embodiments, containing information that enables neuro-circuitry based signatures corresponding to the same subject to be identified. In embodiments in which neuro-circuitry based signature generator 112 generates a neuro-circuitry based signature reflecting a change, neuro-circuitry based signature analyzer may compare that neuro-circuitry based signature to other neuro-circuitry based signatures reflecting change. In other embodiments, neuro-circuitry based signature analyzer 114 may correlate neuro-circuitry based signatures reflecting a patient or patient population in different states and, as part of the neuro-circuitry based signature analysis, determine the change. Neuro-circuitry based signature analyzer 114 may then compare this change in state to reference neuro-circuitry based signatures representing changed states. Alternatively or additionally, neuro-circuitry based signature analyzer 114 may identify reference neuro-circuitry based signatures that characterize subjects or groups of subjects at different times, and compute the change in reference neuro-circuitry based signatures relative to the change in neuro-circuitry based signatures on test subjects output by neuro-circuitry based signature generator 112. Accordingly, it should be appreciated that although the figures illustrate a representative partitioning of processing functions, other partitioning is possible.

As an example of another variation, examples are given of neuro-circuitry based signatures associated with subjects. In some embodiments, the subjects will be humans. In other embodiments, the subjects may alternatively be other animals. Neuro-circuitry based signatures of animals, for example, may be used in a decision support tool configured for making decisions at the pre-clinical stages of an investigation. The decision support tool, for example, may use neuro-circuitry based signatures associated with laboratory animals to indicate pre-clinical measurements to be performed on laboratory to evaluate the efficacy of an experimental drug against a specific CNS condition.

Further, a decision support tool is described in the context of development of drugs. In this context, “drug” should be understood to include any compound administered or considered for administration to a patient to impact a condition or symptoms associated with the condition. Accordingly, a “drug” may include small molecules, biologics, and naturally occurring substances. Alternatively or additionally, techniques described herein may be useful in conjunction with other interventions, including possibly surgical or behavioral interventions, which might be theorized to treat a CNS condition. As a specific example, a system as described herein might be used to assess the effectiveness of medical device that specifically stimulate a specific brain region or neuronal system in the brain. Techniques as described herein may be used to assess whether a specific region targeted with a stimulus signal modifies a neuro-circuitry based signal in a way indicating that a specific condition has been ameliorated, implementing a highly effective biofeedback system, or to assess which region to target with stimulus.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format. In the embodiment illustrated, the input/output devices are illustrated as physically separate from the computing device. In some embodiments, however, the input and/or output devices may be physically integrated into the same unit as the processor or other elements of the computing device. For example, a keyboard might be implemented as a soft keyboard on a touch screen. Alternatively, the input/output devices may be entirely disconnected from the computing device, and functionally integrated through a wireless connection.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A system for predicting the effectiveness of a study compound for treating a CNS condition if the study compound is administered to a patient with the CNS condition, the system comprising:
at least one processor configured to:
access at least one reference neuro-circuitry based signature computed from measurements made on a first plurality of subjects having the CNS condition and treated with reference compounds for which the effectiveness on the CNS condition has been previously determined;
receive measurement data collected during a plurality of measurements of a second plurality of subjects treated with the study compound; and
at least one storage medium storing processor-executable instructions that, when executed by the at least one processor, perform a method comprising:
generating from the measurement data a neuro-circuitry based signature associated with the study compound, the generated neuro-circuitry based signature comprising a plurality of model outputs computed using a plurality of neurocircuit models; and
generating a result indicating effectiveness of the study compound for the CNS condition based at least in part on comparing the generated neuro-circuitry based signature to the at least one reference neuro-circuitry based signature.

2. The system of claim 1, wherein the method further comprises generating, if the result indicates that the study compound is ineffective for treating the CNS condition, an indication of a second CNS condition to evaluate as part of a clinical trial of the study compound, and selecting a set of measurements that provide measurement data for applying a plurality of neurocircuit models associated with the second CNS condition.

3. The system of claim 2, wherein selecting the set of measurements further comprises identifying a plurality of measurements that map to a reference neuro-circuitry based signature associated with the second CNS condition via the plurality of neurocircuit models.

4. The system of claim 1, wherein generating the result includes generating a result indicating whether the study compound alleviates at least one symptom associated with the CNS condition.

5. The system of claim 1, wherein the at least one reference neuro-circuitry based signature is representative of a response to the reference compound of at least one second subject that has the CNS condition.

6. The system of claim 5, wherein the at least one reference neuro-circuitry based signature is indicative of alleviation of at least one symptom of the CNS condition in the at least one second subject in response to administering the reference compound to the second subject.

7. The system of claim 1, wherein generating a result indicating effectiveness comprises comparing the generated neuro-circuitry based signature to the at least one reference neuro-circuitry based signature to compute a level of similarity between the neuro-circuitry based signature and the at least one reference neuro-circuitry based signature.

8. The system of claim 1, wherein generating a result indicating effectiveness further comprises selecting a portion of the neurocircuits of the plurality of neurocircuits that correspond to brain functions indicative of the CNS condition and comparing model outputs associated with the portion of the neurocircuits between the neuro-circuitry based signature and the at least one reference neuro-circuitry based signature.

9. A system for predicting at least one CNS condition treatable with a study compound if the study compound is administered to a patient with the at least one CNS condition, the system comprising:
at least one processor configured to:
access at least one reference neuro-circuitry based signature computed from measurements made on a first plurality of subjects having a plurality of CNS conditions and treated with at least one reference compound that has been previously determined to be effective in treating a CNS condition;
receive measurement data collected during a plurality of measurements of at least one second subject to whom the study compound has been administered; and
at least one storage medium storing processor-executable instructions that, when executed by the at least one processor, perform a method comprising:
generating a neuro-circuitry based signature associated with the study compound, the generated neuro-circuitry based signature comprising a plurality of model outputs generated by computing, using a plurality of neurocircuit models, the plurality of model outputs from the measurement data; and
indicating at least one CNS condition by:
comparing the generated neuro-circuitry based signature to the at least one reference neuro-circuitry based signature and,
based on a degree of similarity between the generated neuro-circuitry based signature and a reference neuro-circuitry based signature of the at least one reference neuro-circuitry based signature associated with effective treatment of a known CNS condition, indicating that the study compound is predicted to be effective in treating the known CNS condition.

10. The system of claim 9, wherein the method further comprises identifying at least one domain of brain function affected by the compound by determining a portion of model outputs of the plurality of model outputs that describe the response of the subject to the compound based on the degree of similarity between the generated neuro-circuitry based signature and the reference neuro-circuitry based signature.

11. The system of claim 10, wherein the method further comprises identifying at least one neurocircuit among the plurality of neurocircuits associated with the portion of model outputs.

12. The system of claim 10, wherein the method further comprises identifying the at least one CNS condition based on the at least one domain of brain function.

13. The system of claim 9, wherein comparing the generated neuro-circuitry based signature to the at least one reference neuro-circuitry based signature comprises computing a correlation value between the generated neuro-circuitry based signature and the at least one reference neuro-circuitry based signature and comparing the correlation value to a threshold.

14. A system for designing a clinical trial for a study compound, the system comprising:
at least one processor configured to:
access at least one reference neuro-circuitry based signature computed from a plurality of measurements made on a first plurality of subjects having a CNS condition and treated with reference compounds that have previously been shown to be effective at treating a CNS condition, wherein each of the at least one reference neuro-circuitry based signature has associated therewith a plurality of neurocircuit models from which the reference neuro-circuitry based signature is generated based on the plurality of measurements;
receive a study neuro-circuitry based signature representative of a second plurality of subjects' response to the study compound, wherein the study neuro-circuitry based signature includes a plurality of model outputs computed, using neurocircuitry models, from measurements made on the second plurality of subjects before and after treatment with the study compound; and
at least one non-transitory storage medium storing processor-executable instructions that, when executed by the at least one processor, perform a method comprising:
comparing the study neuro-circuitry based signature to the at least one reference neuro-circuitry based signature;
based on a degree of similarity between the study neuro-circuitry based signature and a selected reference neuro-circuitry based signature of the at least one reference neuro-circuitry based signature, selecting a set of measurements that provide measurement data for applying the plurality of neurocircuit models associated with the selected reference neuro-circuitry based signature.

15. The system of claim 14, wherein the method further comprises generating a result indicating at least one CNS condition to evaluate as part of the clinical trial, wherein the at least one CNS condition is associated with at least one domain of brain function.

16. The system of claim 15, wherein the method further comprises generating a result indicating at least one symptom to identify in subjects as part of enrollment in the clinical trial, wherein the at least one symptom is associated with the at least one CNS condition.

17. The system of claim 16, wherein the method further comprises identifying at least one type of equipment for obtaining the measurement data during the clinical trial.

18. A system for designing a clinical trial, the system comprising:
at least one processor configured to:
receive user input indicating a CNS condition to evaluate as part of the clinical trial; and
access a plurality of reference neuro-circuitry based signatures;
at least one storage medium storing processor-executable instructions that, when executed by the at least one processor, perform a method comprising:
selecting at least one reference neuro-circuitry based signature of the plurality of reference neuro-circuitry based signatures, the at least one selected reference neuro-circuitry based signature being computed from a plurality of measurements made on a first plurality of subjects having the CNS condition and treated with reference compounds for which the effectiveness on the condition has been previously determined, wherein each of the at least one reference neuro-circuitry based signature has associated therewith a plurality of neurocircuit models that map the plurality of measurements to the reference neuro-circuitry based signature; and
generating, based on the plurality of measurements mapped to the at least one reference neuro-circuitry based signature via the neurocircuit models of the at least one selected reference neuro-circuitry based signature, a result indicating a plurality of measurements to include in a protocol for the clinical trial.

19. The system of claim 18, wherein the method further comprises identifying a portion of the neurocircuit models of the plurality of neurocircuit models that correspond to brain functions indicative of the CNS condition.

20. The system of claim 19, wherein the plurality of measurements to include in the protocol of the clinical trial provide data indicative of the brain functions.
